(19) United States
(12) Patent Application Publication
(10) Pub. No.: US 2026/0060693 A1
(43) Pub. Date: Mar. 5, 2026

(54) NON-RATCHETING SURGICAL CLIP APPLICATION DEVICE

(71) Applicants: Baxter International Inc., Deerfield, IL (US); Baxter Healthcare SA, Glattpark (Opfikon) (CH)

(72) Inventors: Kyle Stewart, Cincinnati, OH (US); Shannon Mary Witkowski, St. Paul, MN (US)

(21) Appl. No.: 19/311,582

(22) Filed: Aug. 27, 2025

Related U.S. Application Data
(60) Provisional application No. 63/687,503, filed on Aug. 27, 2024.

(51) Int. Cl. A61B 17/128; A61B 17/00

(52) U.S. Cl. CPC A61B 17/128; A61B 2017/00367

(57) ABSTRACT

A non-ratcheting surgical clip application device for applying and crimping surgical clips to ligate a vessel. The surgical clip applying device includes an enclosure formed from a top housing, a bottom housing and a window cover, a pair of handles including a left handle and a right handle each mounted for pivotal movement in the bottom housing, a main rail fixedly mounted to the bottom housing within the enclosure, a pair of clip applying jaws fixedly attached to the bottom housing and mounted through the main rail, a jaw closer comprising a proximal end, a distal end, and an elongate base, and a drive mechanism including a plurality of levers and at least one spring.

### CROSS-REFERENCE TO RELATED APPLICATIONS

The present application claims the benefit of U.S. Provisional Application No. 63/687,503, entitled NON-RATCHETING SURGICAL CLIP APPLICATION DEVICE and filed Aug. 27, 2024, the contents of which are hereby incorporated by reference in their entirety.

### BACKGROUND

Clip application devices or clip appliers may be used during microsurgery procedures. Microsurgery covers a variety of procedures using a microscope for operating on successively smaller blood vessels, nerves, etc. Conventional suturing techniques in microsurgery have given way to the use of surgical clips, which are applied at the end of a severed vessel or at a junction of vessels. For example, the surgical clips hold or close the tissue in a similar fashion as conventional sutures. The clips used in microsurgery are very small and are typically called microclips. An example microclip may have a size on the order of 2 mm high and 2 mm wide.

Microclips are commonly applied in surgery by forceps that load and use a single clip at a time. Single load-and-use devices are burdensome to medical professionals, as several clips may be required for a surgical procedure. The numerous reloading actions may cause handling accidents (e.g., dropping a clip on the floor, dropping an instrument, dropping a closed or partially closed clip into a wound), misloaded clips, etc. Additionally, using several sets of forceps requires cleaning and otherwise maintaining a stock of several forceps.

The present disclosure is directed to a non-ratcheting surgical microclip applier for use in microsurgery that provides significant advantages over prior art instruments of the type described above.

### SUMMARY

The present disclosure provides a surgical clip application device or clip applier for applying surgical clips to ligate vessels during surgery. The clip applier disclosed herein may automatically load clips, which allows a user to ligate multiple vessels during surgery. As a user aligns a vessel within the jaws of the device and squeezes the device handle, a surgical clip is crimped to ligate the vessel aligned within the jaws. Once the handle is released, another clip is automatically loaded and ready for ligating another vessel.

In an example, a surgical clip applying device includes an enclosure formed from a top housing, a bottom housing and a window cover. The clip applying device also includes a pair of handles including a left handle and a right handle, a main rail supporting a jaw closer, and a pair of clip applying jaws fixedly attached to the bottom housing and retained within the main rail. The pair of clip applying jaws has arms terminating with a pair of jaw heads. Additionally, the clip applying device includes a clip loader assembly with a clip loading tip at the clip loader assembly's distal end and a dispenser positioned above the clip loader assembly. The dispenser includes a pair of clip forks and a retention prong positioned between the pair of clip forks at the dispenser's distal end. The clip loading tip is configured to push a surgical clip supported by the pair of clip forks over the retention prong. The clip applying device also includes a clip pusher assembly configured to advance a plurality of clips towards the pair of jaw heads and a drive mechanism including a plurality of levers and at least one spring. The drive mechanism is configured to translate an actuation of the pair of handles to a non-ratcheting linear motion of the jaw closer and the clip loader assembly.

In some aspects, the techniques described herein relate to a surgical clip applying device including: an enclosure formed from a top housing, a bottom housing and a window cover; a pair of handles including a left handle and a right handle each mounted for pivotal movement in the bottom housing; a main rail fixedly mounted to the bottom housing within the enclosure; a pair of clip applying jaws fixedly attached to the bottom housing and mounted through the main rail; a jaw closer including a proximal end, a distal end, and an elongate base; and a drive mechanism including a plurality of levers and at least one spring.

In some aspects, which may be used in combination with any other aspect disclosed herein, the enclosure includes an elongate base and upstanding sidewalls defining a U-shaped channel.

In some aspects, which may be used in combination with any other aspect disclosed herein, the pair of clip applying jaws include at least one arm, wherein the at least one arm terminates at a pair of spaced opposing jaw heads.

In some aspects, which may be used in combination with any other aspect disclosed herein, the pair of clip applying jaws include a pair of angled ramp surfaces disposed in said arms.

In some aspects, which may be used in combination with any other aspect disclosed herein, the arms are spaced inwardly from said jaw heads.

In some aspects, which may be used in combination with any other aspect disclosed herein, the distal end of the jaw closer includes a V-shaped indent.

In some aspects, which may be used in combination with any other aspect disclosed herein, the proximal end of the jaw closer includes a pair of upstanding drive flanges.

In some aspects, which may be used in combination with any other aspect disclosed herein, the device includes an elongate slot at a distance from the distal end of the jaw closer and at a distance from the proximal end of the jaw closer.

In some aspects, which may be used in combination with any other aspect disclosed herein, the jaw closer is disposed at a distance between the clip applying jaws and at a distance between the base of the rail.

In some aspects, which may be used in combination with any other aspect disclosed herein, the jaw closer is mounted for slideable movement in the channel toward and away from the jaw heads.

In some aspects, which may be used in combination with any other aspect disclosed herein, the device includes cam members configured to engage and compress a first ramp ramp surface and a second ramp surface, wherein the first ramp surface and the second ramp surface are disposed on the arms of the clip applying jaws.

In some aspects, which may be used in combination with any other aspect disclosed herein, the cam members are configured to force the arms and the jaw heads together as the jaw closer is advanced towards the jaw heads.

In some aspects, which may be used in combination with any other aspect disclosed herein, connecting the handles are engaged with the upstanding drive flanges of the jaw closer.

In some aspects, which may be used in combination with any other aspect disclosed herein, the drive mechanism is configured to translate an actuation of the pair of handles to a non-ratcheting linear motion of the jaw closer.

In some aspects, which may be used in combination with any other aspect disclosed herein, the techniques described herein relate to a clip applier surgery system including a clip applier, wherein the clip applier includes: an enclosure formed from a top housing, a bottom housing and a window cover; a pair of handles including a left handle and a right handle each mounted for pivotal movement in the bottom housing; a main rail fixedly mounted to the bottom housing within the enclosure; a pair of clip applying jaws fixedly attached to the bottom housing and mounted through the main rail; a jaw closer including a proximal end, a distal end, and an elongate base; and a drive mechanism including a plurality of levers and at least one spring.

In some aspects, which may be used in combination with any other aspect disclosed herein, the techniques described herein relate to a method of ligating vessels during surgery using a surgical clip applying device, wherein the surgical applying device includes: an enclosure formed from a top housing, a bottom housing and a window cover; a pair of handles including a left handle and a right handle each mounted for pivotal movement in the bottom housing; a main rail fixedly mounted to the bottom housing within the enclosure; a pair of clip applying jaws fixedly attached to the bottom housing and mounted through the main rail; a jaw closer including a proximal end, a distal end, and an elongate base; and a drive mechanism including a plurality of levers and at least one spring.

In some aspects, which may be used in combination with any other aspect disclosed herein, the enclosure includes an elongate base and upstanding sidewalls defining a U-shaped channel.

In some aspects, which may be used in combination with any other aspect disclosed herein, the pair of clip applying jaws include at least one arm, wherein the at least one arm terminates at a pair of spaced opposing jaw heads.

In some aspects, which may be used in combination with any other aspect disclosed herein, the pair of clip applying jaws include a pair of angled ramp surfaces disposed in said arms.

In some aspects, which may be used in combination with any other aspect disclosed herein, the arms are spaced inwardly from said jaw heads.

The surgical clip application device or clip applier disclosed herein is expected to offer convenience compared to devices and systems that require manually loading individual clips.

It is another advantage of the present disclosure to provide a surgical clip application device (e.g., clip applier) with automatic surgical clip reloading.

It is another advantage of the present disclosure to provide non-ratcheting actuation thereby allowing the jaws to be temporarily released and/or reduce forces or pressure applied to the surgical clip such that the clip and/or clip applier may be repositioned and reoriented during use.

Additionally, another advantage of the present disclosure is to provide a surgical clip application device (e.g., clip applier) with a transparent window thereby allowing a user to easily ascertain a remaining clip count for surgical clips housed within the device.

It is a further advantage of the present disclosure to provide a surgical clip application device (e.g., clip applier) with multiple touch points, finger loops, and gripping surfaces for ambidextrous surgical clip application.

Additional features and advantages of the disclosed surgical clip application device (e.g., clip applier), systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

FIG. 1A is a top view of a clip application device, according to one or more examples of the present disclosure.

FIG. 1B is a side view of the clip application device of FIG. 1A, according to one or more examples of the present disclosure.

FIG. 1C is an exploded perspective view of the clip application device of FIG. 1A, according to one or more examples of the present disclosure.

FIG. 1D is a partial top view, with the top housing removed, of the clip application device of FIG. 1A, according to one or more examples of the present disclosure.

FIG. 1E is a cross-sectional view along line A-A of FIG. 1D, according to one or more examples of the present disclosure.

FIG. 2A is an exploded perspective view of a bottom closure assembly, according to one or more examples of the present disclosure.

FIG. 2B is a top view of a bottom closure assembly, according to one or more examples of the present disclosure.

FIG. 2C is a cross-sectional view along line A-A of FIG. 2B, according to one or more examples of the present disclosure.

FIG. 3A is an exploded perspective view of a right handle assembly, according to one or more examples of the present disclosure.

FIG. 3B is a top view of a right handle assembly, according to one or more examples of the present disclosure.

FIG. 3C is a cross-sectional view along line A-A of FIG. 3B, according to one or more examples of the present disclosure.

FIG. 4 is an exploded perspective view of a left handle assembly, according to one or more examples of the present disclosure.

FIG. 5A is an exploded perspective view of a clip dispenser assembly, according to one or more examples of the present disclosure.

FIG. 5B is a top view of a clip dispenser assembly, according to one or more examples of the present disclosure.

FIG. 5C is a side view of a clip dispenser assembly, according to one or more examples of the present disclosure.

FIG. 6A is a side view of a bottom housing, according to one or more examples of the present disclosure.

FIG. 6B is a bottom view of a bottom housing, according to one or more examples of the present disclosure.

FIG. 6C is a cross-sectional view along line A-A of FIG. 6B, according to one or more examples of the present disclosure.

FIG. 6D is a cross-sectional view along line F-F of FIG. 6B, according to one or more examples of the present disclosure.

FIG. 6E is a cross-sectional view along line C-C of FIG. 6B, according to one or more examples of the present disclosure.

FIG. 6F is a cross-sectional view along line D-D of FIG. 6B, according to one or more examples of the present disclosure.

FIG. 7A is a perspective view of a main rail, according to one or more examples of the present disclosure.

FIG. 7B is a side view of a main rail, according to one or more examples of the present disclosure.

FIG. 7C is a top view of a main rail, according to one or more examples of the present disclosure.

FIG. 7D is a cross-sectional view along line B-B of FIG. 7C, according to one or more examples of the present disclosure.

FIG. 7E is a rear view of a main rail, according to one or more examples of the present disclosure.

FIG. 8A is a perspective view of a pair of clip applying jaws, according to one or more examples of the present disclosure.

FIG. 8B is a top view of a pair of clip applying jaws in an open configuration, according to one or more examples of the present disclosure.

FIG. 8C is a side view of a pair of clip applying jaws, according to one or more examples of the present disclosure.

FIG. 8D is a top view of a pair of clip applying jaws, according to one or more examples of the present disclosure.

FIG. 8E is a cross-sectional view along line A-A of FIG. 8D, according to one or more examples of the present disclosure.

FIG. 8F is a bottom view of a pair of clip applying jaws in a closed configuration, according to one or more examples of the present disclosure.

FIG. 8G is a partial detail view of the portion of the clip applying jaws from FIG. 8E, according to one or more examples of the present disclosure.

FIG. 8H is a cross-sectional view along line D-D of FIG. 8E, according to one or more examples of the present disclosure.

FIG. 8J is a partial detail view of the portion of the clip applying jaws from FIG. 8E, according to one or more examples of the present disclosure.

FIG. 9A is a perspective view of a jaw closer, according to one or more examples of the present disclosure.

FIG. 9B is a top view of a jaw closer, according to one or more examples of the present disclosure.

FIG. 9C is a side view of a jaw closer, according to one or more examples of the present disclosure.

FIG. 9D is a partial detail view of the portion of the jaw closer from FIG. 9B, according to one or more examples of the present disclosure.

FIG. 9E is a partial detail view of the portion of the jaw closer from FIG. 9C, according to one or more examples of the present disclosure.

FIG. 10A is a top view of a dispenser, according to one or more examples of the present disclosure.

FIG. 10B is a side view of a dispenser, according to one or more examples of the present disclosure.

FIG. 10C is a cross-sectional view along line J-J of FIG. 10A, according to one or more examples of the present disclosure.

FIG. 10D is a cross-sectional view along line G-G of FIG. 10A, according to one or more examples of the present disclosure.

FIG. 10E is a rear view of a dispenser, according to one or more examples of the present disclosure.

FIG. 10F is a cross-sectional view along line K-K of FIG. 10A, according to one or more examples of the present disclosure.

FIG. 10G is a partial detail view of the portion of the dispenser from FIG. 10A, according to one or more examples of the present disclosure.

FIG. 10H is a partial detail view of the portion of the dispenser from FIG. 10C, according to one or more examples of the present disclosure.

FIG. 11A is a perspective view of a clip loader assembly, according to one or more examples of the present disclosure.

FIG. 11B is a perspective view of a distal clip loader, according to one or more examples of the present disclosure.

FIG. 11C is a top view of a distal clip loader, according to one or more examples of the present disclosure.

FIG. 11D is a side view of a distal clip loader, according to one or more examples of the present disclosure.

FIG. 11E is a cross-sectional view along line R-R of FIG. 11C, according to one or more examples of the present disclosure.

FIG. 11F is a cross-sectional view along line AA-AA of FIG. 11E, according to one or more examples of the present disclosure.

FIG. 12A is a perspective view of a proximal clip loader, according to one or more examples of the present disclosure.

FIG. 12B is a top view of a proximal clip loader, according to one or more examples of the present disclosure.

FIG. 12C is a cross-sectional view along line E-E of FIG. 12B, according to one or more examples of the present disclosure.

FIG. 12D is a partial detail view of the portion of the proximal clip loader from FIG. 12C, according to one or more examples of the present disclosure.

### DETAILED DESCRIPTION OF EXAMPLE EMBODIMENTS

The surgical clip application device (e.g., clip applier) for ligating vessels described herein provides improved surgical clip application, automatic surgical clip reloading, and safety lock-out features.

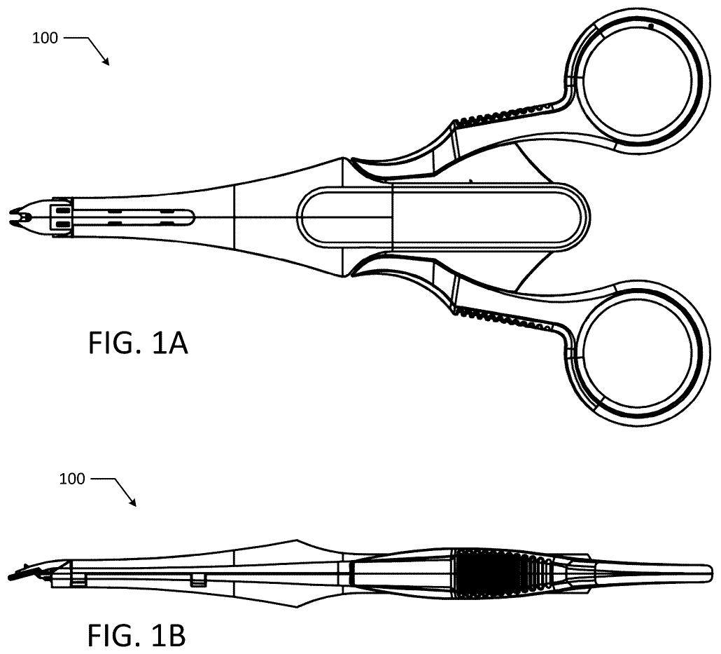

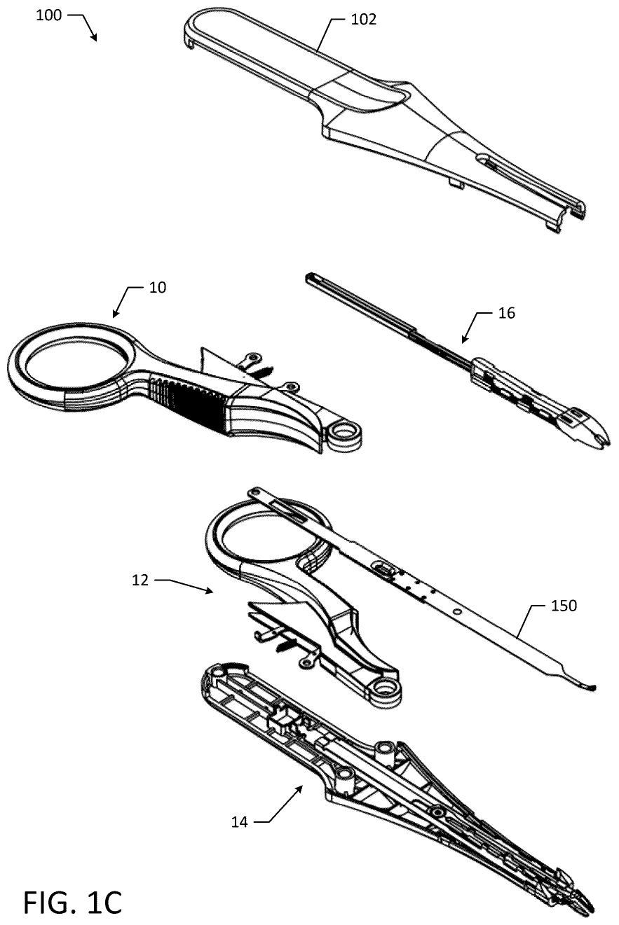

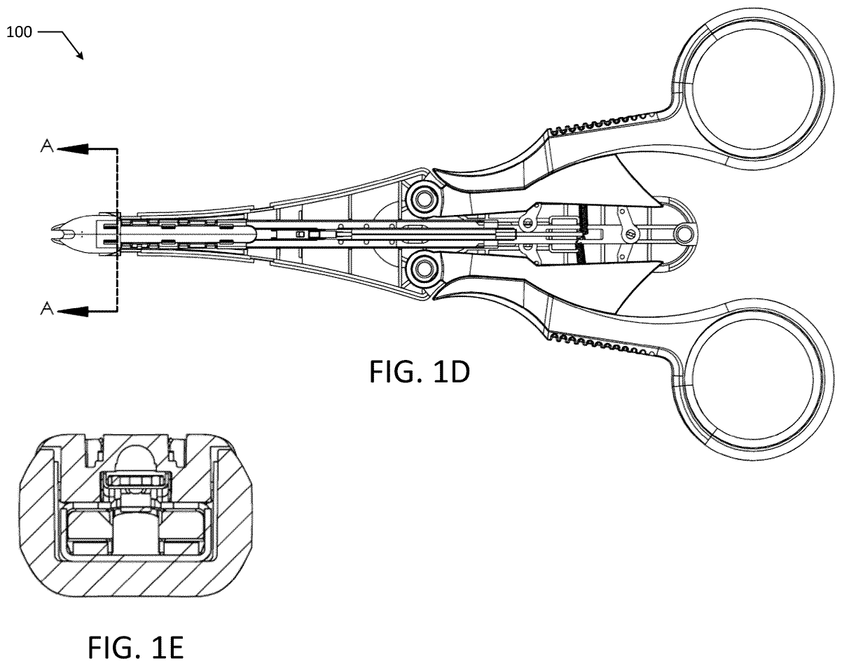

FIGS. 1A, 1B, IC, 1D and 1E illustrate an example embodiment of a clip applier 100. The clip applier 100 is configured to apply clips to a surgical site. Similar to clip applier 100 described in U.S. patent application Ser. No. 17/901,463, entitled “Surgical Clip Application Device”, the entire contents of which are incorporated by reference herein, the clip applier 100 includes a top housing 102, a bottom housing 104, a left handle 106a, and a right handle 106b that together form an enclosure for the clip applier 100. It should be appreciated that the various components of chip applier 100 described herein may share the same or similar features of the components of the various chip applier embodiments described in U.S. patent application Ser. No. 17/901,463. For example, clip loader assembly 150 illustrated in FIG. 1C may include the same features of clip loader assembly 150 as described in U.S. patent application Ser. No. 17/901,463. More generally speaking, the features and components of chip applier 100 may have the same or similar construction and features as the chip applier examples described in U.S. patent application Ser. No. 17/901,463.

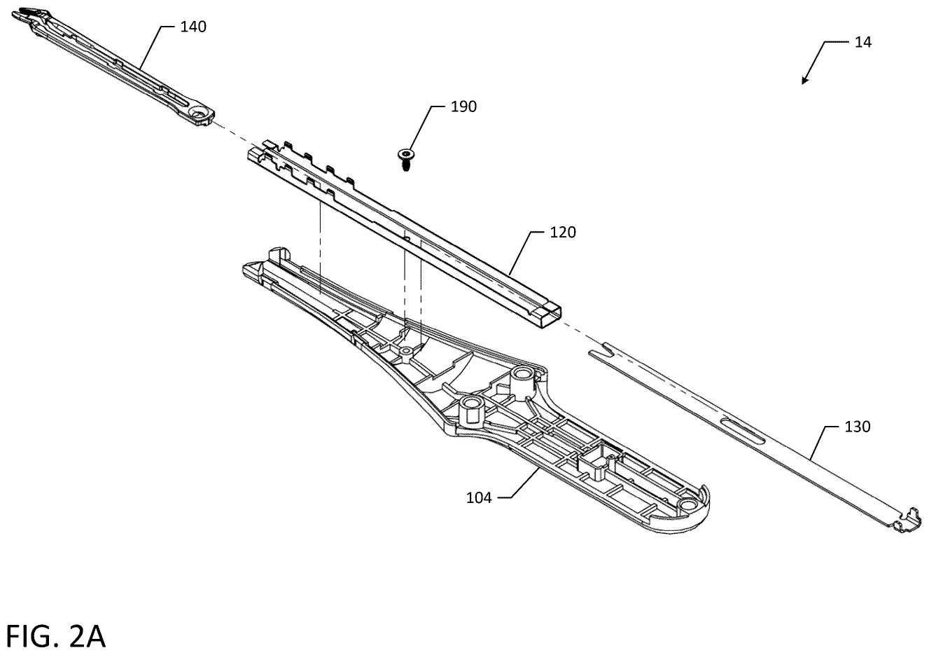

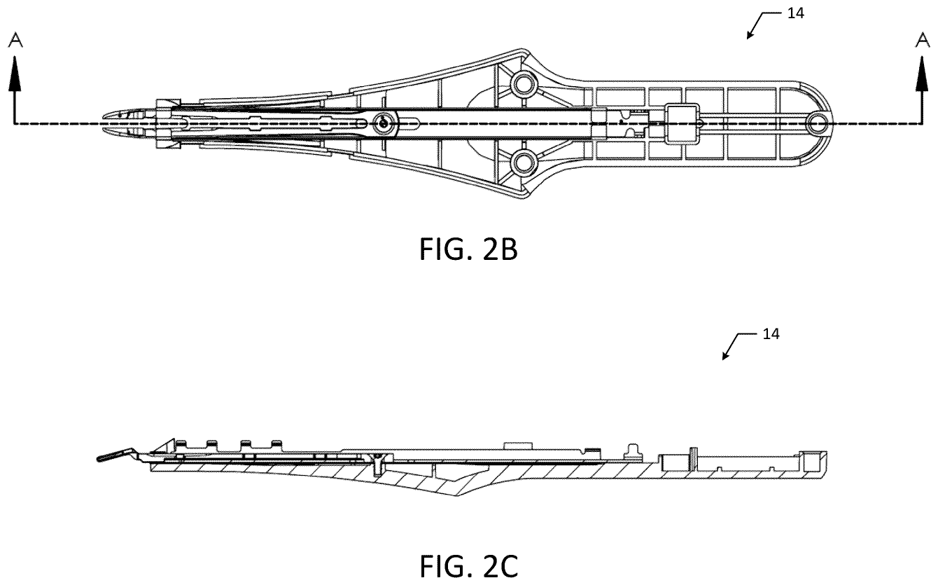

FIGS. 2A-2C illustrate an example bottom enclosure assembly 14. The features and components of bottom enclosure assembly 14 may have the same or similar construction and features as the bottom enclosure assembly examples described in U.S. patent application Ser. No. 17/901,463 (see FIG. 1J and corresponding description of U.S. patent application Ser. No. 17/901,463). The bottom enclosure assembly 14 may include a bottom housing 104, a main rail 120, a jaw closer 130, a pair of clip applying jaws 140, and a jaw anchor 190.

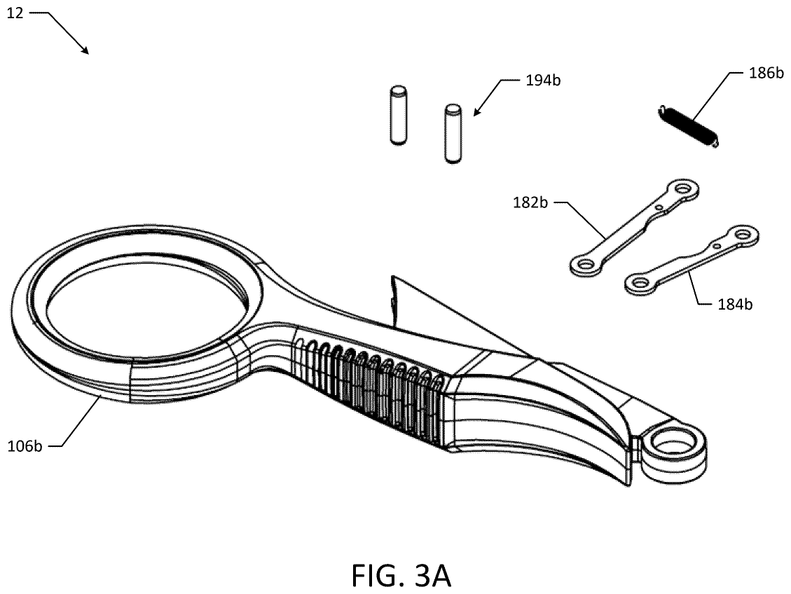

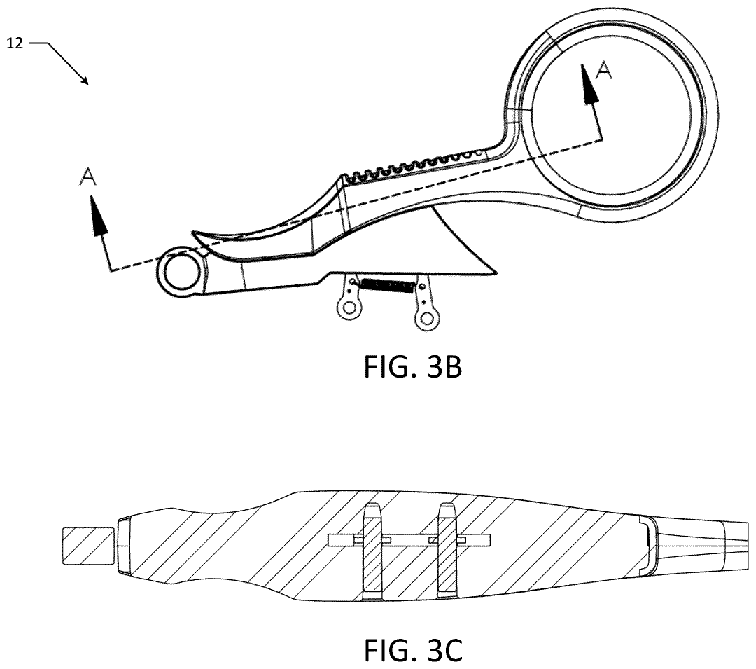

FIGS. 3A-3C illustrate an example right handle assembly 12. The features and components of right handle assembly 12 may have the same or similar construction and features as the right handle assembly examples described in U.S. patent application Ser. No. 17/901,463 (see FIG. 1I and corresponding description of U.S. patent application Ser. No. 17/901,463). The right handle assembly 12 may include a right handle 106b, a proximal lever 182b, a distal lever 184b, a spring 186b, and pins 194b.

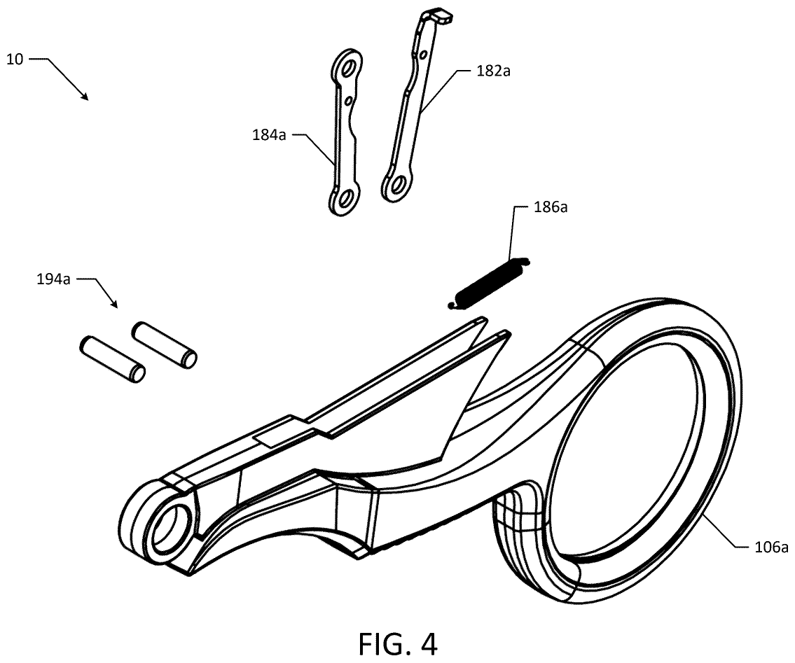

FIG. 4 illustrates an example left handle assembly 10. The features and components of left handle assembly 10 may have the same or similar construction and features as the left handle assembly examples described in U.S. patent application Ser. No. 17/901,463 (see FIG. 1H of U.S. patent application Ser. No. 17/901,463). The left handle assembly 10 may include a left handle 106a, a proximal lever 182a, a distal lever 184a, a spring 186a, and pins 194a.

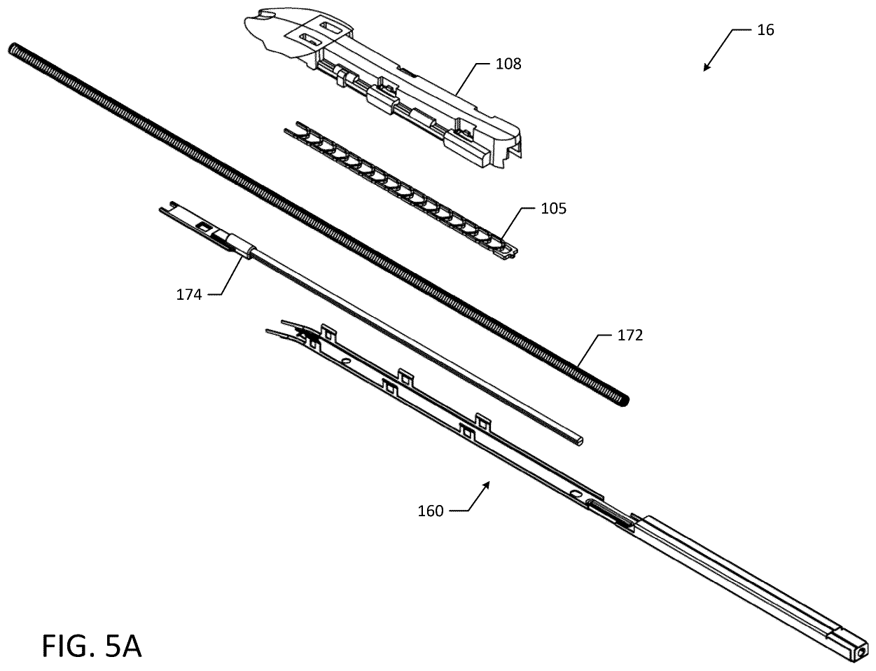

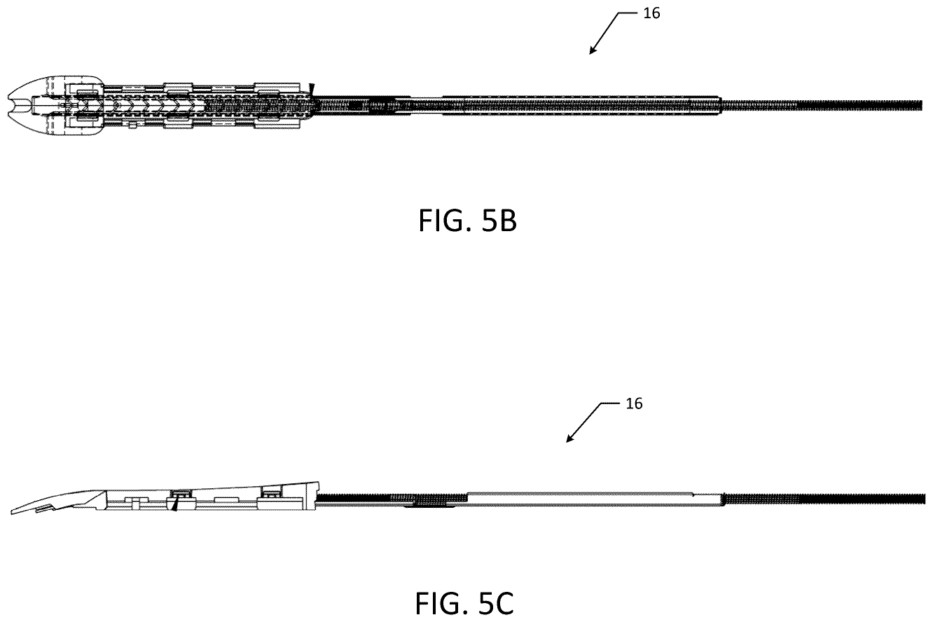

FIGS. 5A-5C illustrate an example clip dispenser assembly 16. The features and components of clip dispenser assembly 16 may have the same or similar construction and features as the clip dispenser assembly examples described in U.S. patent application Ser. No. 17/901,463 (see FIG. 1K and corresponding description of U.S. patent application Ser. No. 17/901,463). The clip dispenser assembly 16 may include a stack 105 of surgical clips, a window cover 108, a dispenser 160, a clip pusher spring 172, and a clip pusher bar 174.

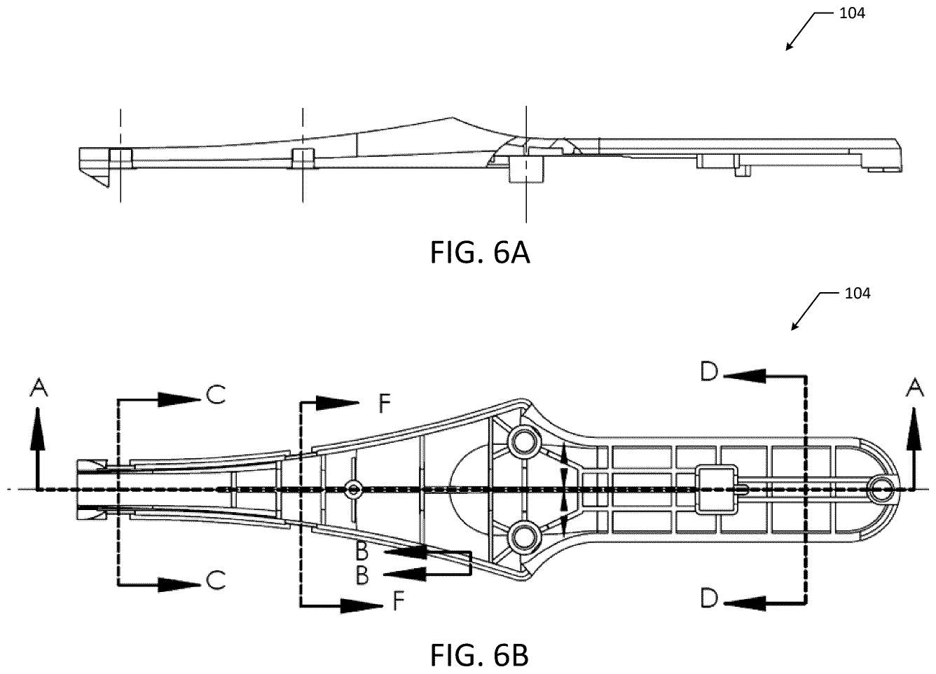

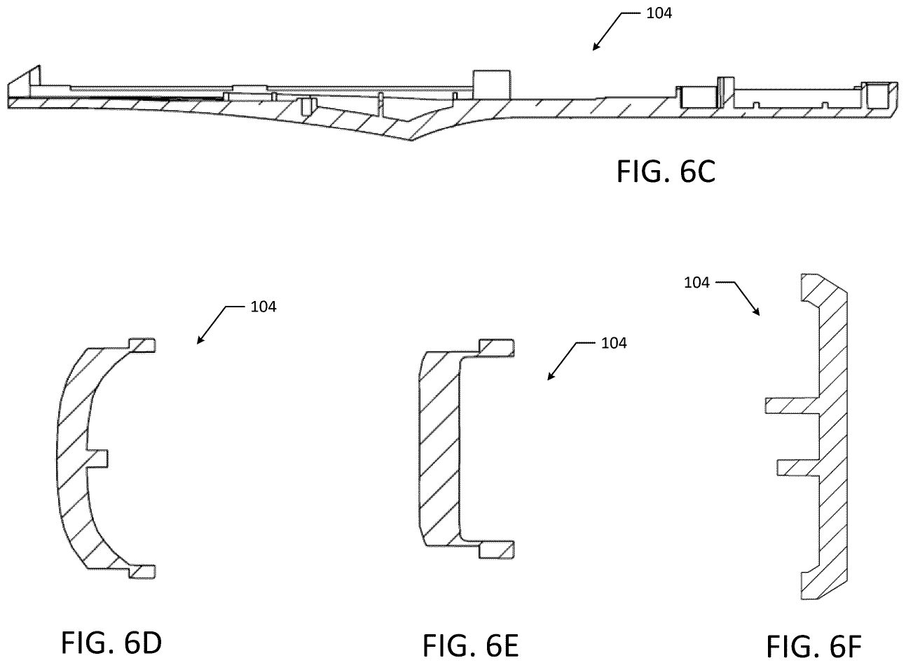

FIGS. 6A-6F illustrate an example bottom housing 104. The features and components of bottom housing 104 may have the same or similar construction and features as the bottom housing examples described in U.S. patent application Ser. No. 17/901,463.

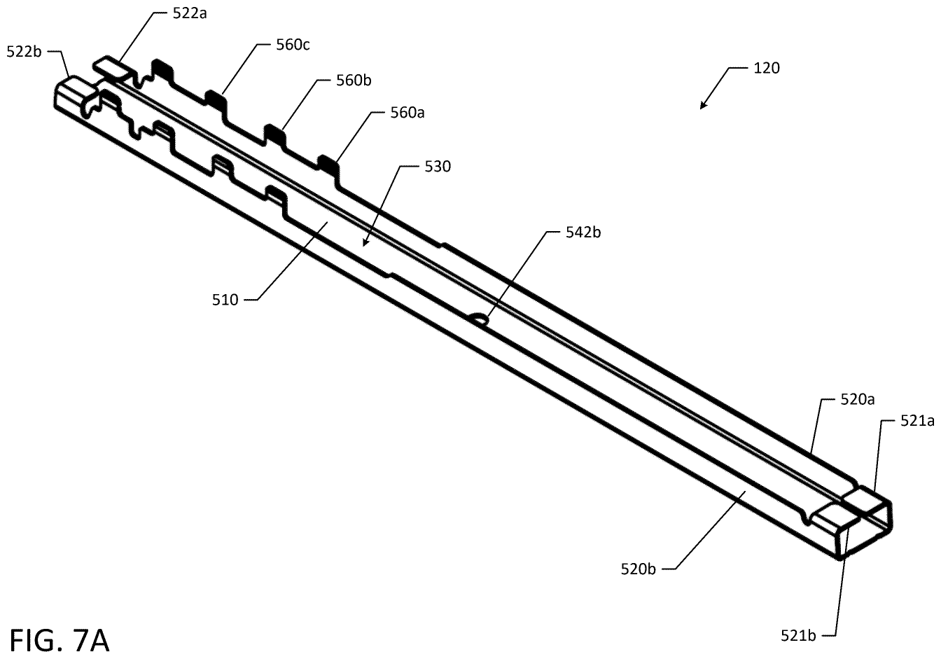

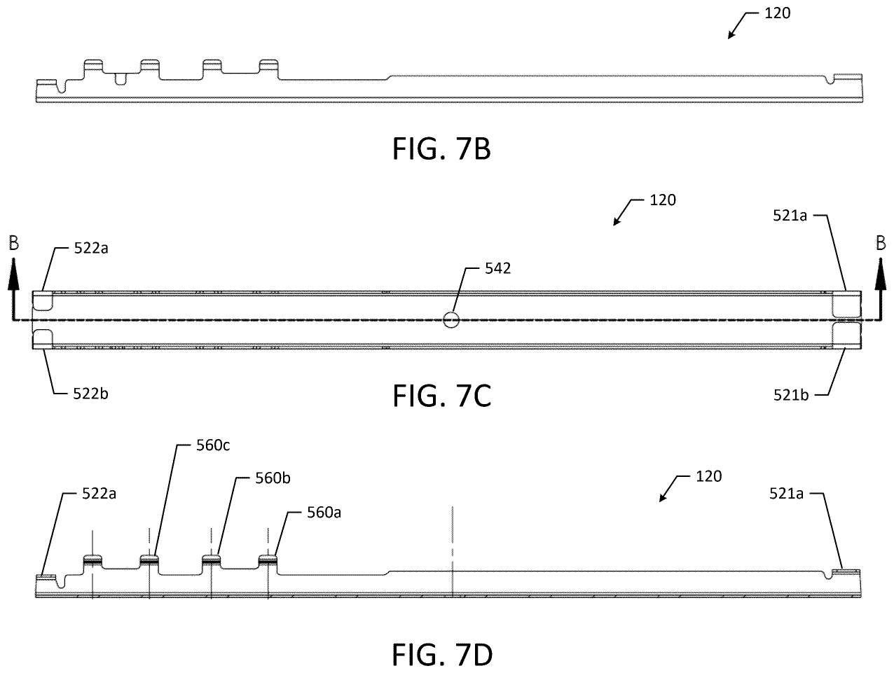

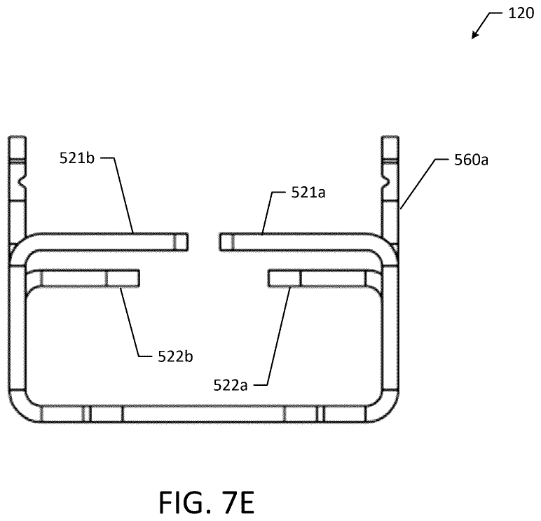

FIGS. 7A-7E illustrate an example main rail 120. The features and components of main rail 120 may have the same or similar construction and features as the main rail examples described in U.S. patent application Ser. No. 17/901,463 (see FIG. 5G and corresponding description of U.S. patent application Ser. No. 17/901,463). The main rail may include a base 510, sidewalls 520a and 520b that form channel 530, and hole(s) 542b. The main rail 120 may also include a plurality of elongate members (hereinafter referred to as elongate members 560) exemplified by elongate members 560a, 560b, and 560c. In some examples, the elongate members 560 can be used to mechanically couple the main rail 120 to a window cover (e.g., the window cover 108 of FIG. 1K of U.S. patent application Ser. No. 17/901,463).

Additionally, the side walls 520a and 520b, hereinafter referred to generally as side walls 520, may be folded over forming flanges 522a and 522b. The flanges, which are referred to generally as flanges 522 are adapted to constrain the interaction of the clip applying jaws 140 and the jaw closer 130 thereby preventing the clip applying jaws 140 and the jaw closer 130 from disengaging. Similarly, the opposite end of the main rail may include flanges 521a and 521b, which may also be formed from folding over the sidewalls and may be configured to retain, house or prevent disengagement of or between various components of the chip applier 100.

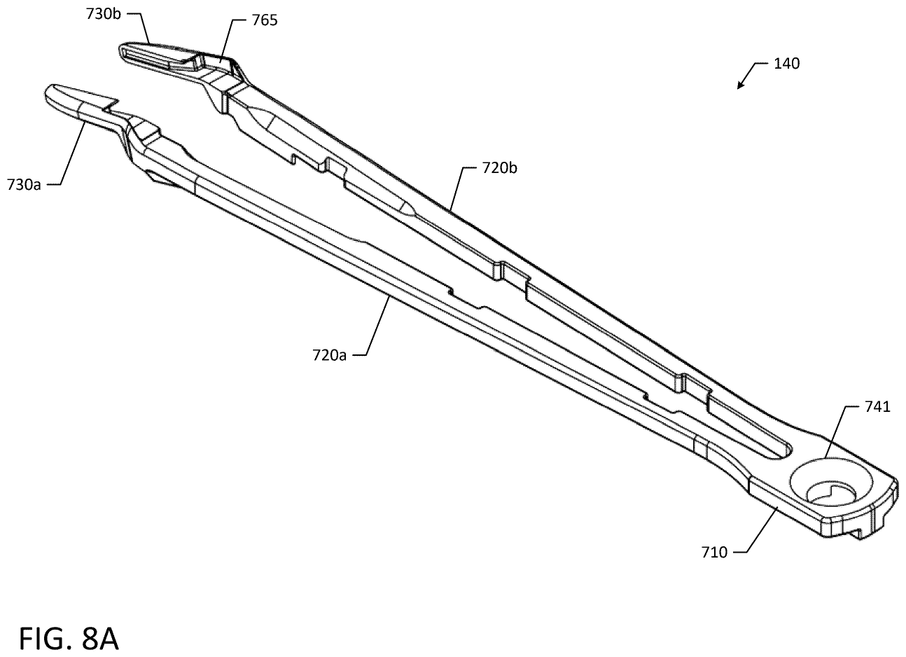

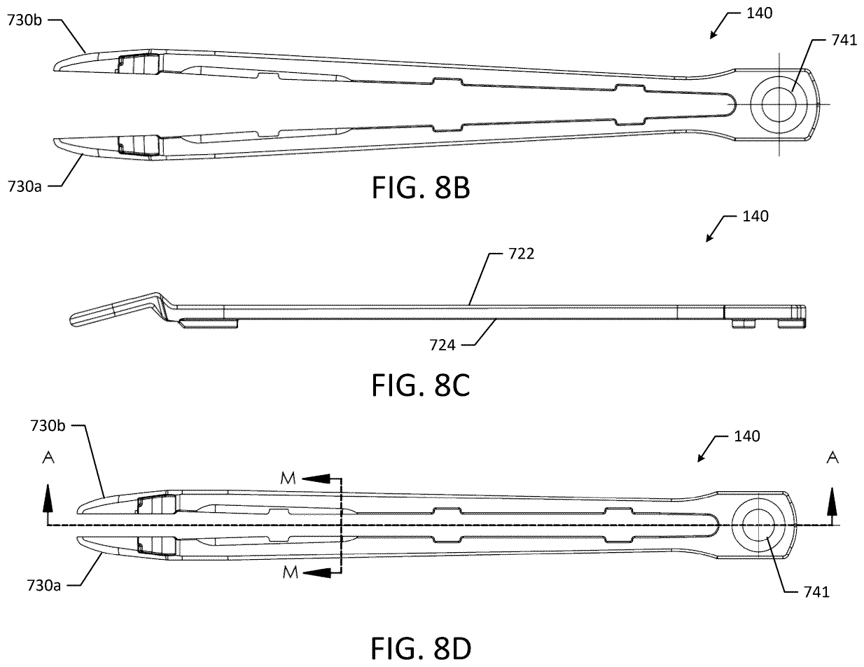

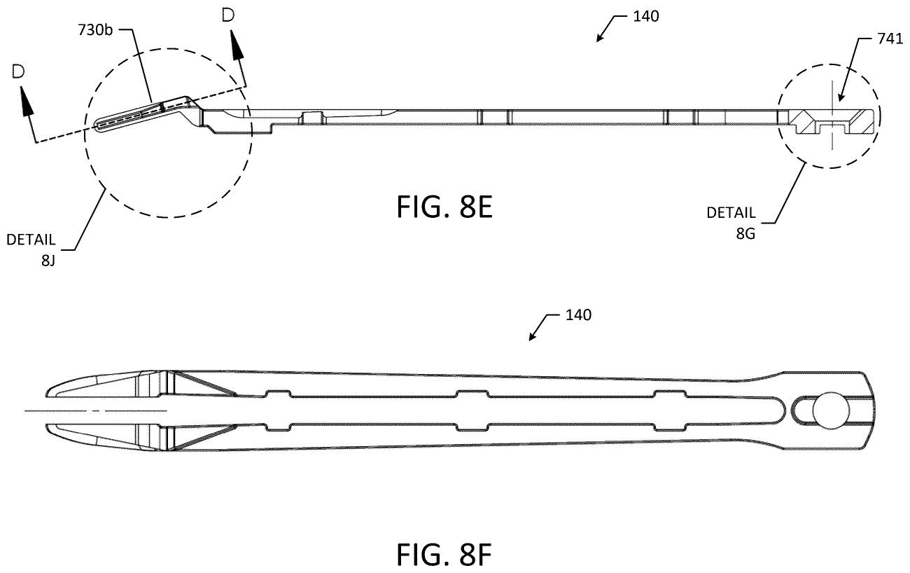

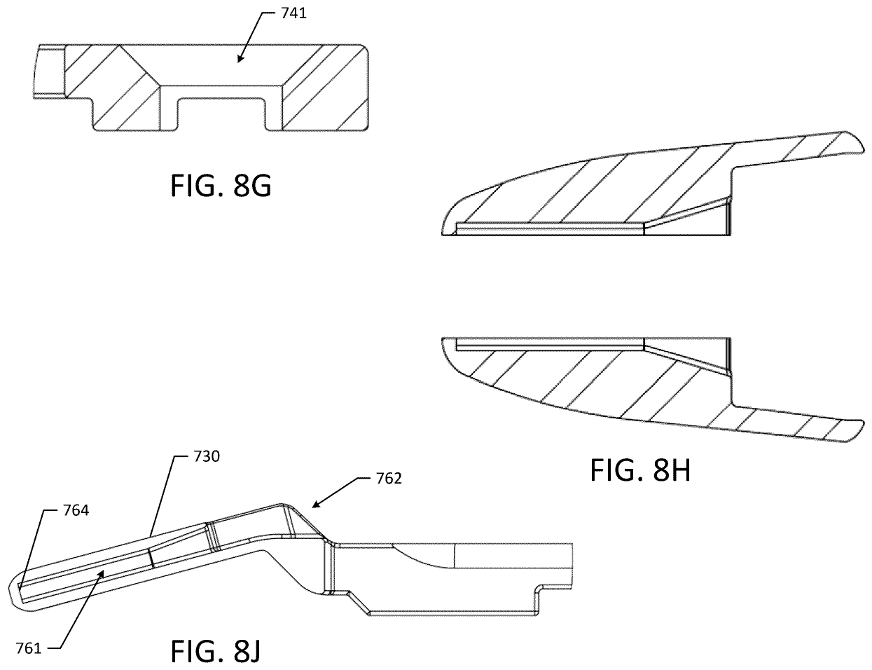

FIGS. 8A-8J illustrate an example pair of clip applying jaws 140. The features and components of clip applying jaws 140 may have the same or similar construction and features as the clip applying jaws examples described in U.S. patent application Ser. No. 17/901,463 (see FIGS. 7A-7F and corresponding description of U.S. patent application Ser. No. 17/901,463). The clip applying jaws 140 may include a body portion 710 at a proximal end and arms 720a, 720b (e.g., right arm 720a and left arm 720b) that extend from the body portion 710 towards a distal end of the clip applying jaws 140. Additionally, the clip applying jaws 140 may include an opening or thru-hole 741 that accommodates jaw anchor 190. The arms 720a and 720b, hereinafter referred to generally as arms 720 terminate in cooperating jaw heads 730a, 730b at the distal end 703.

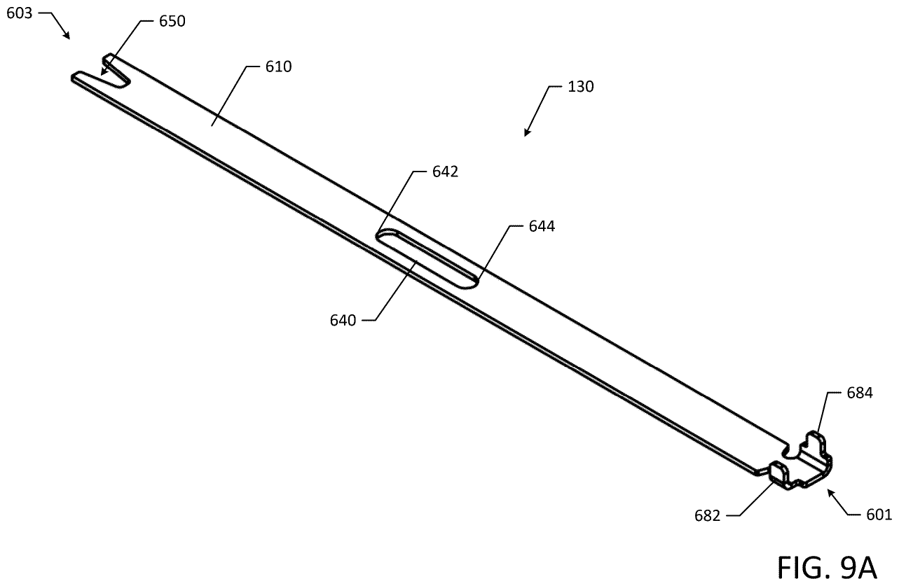

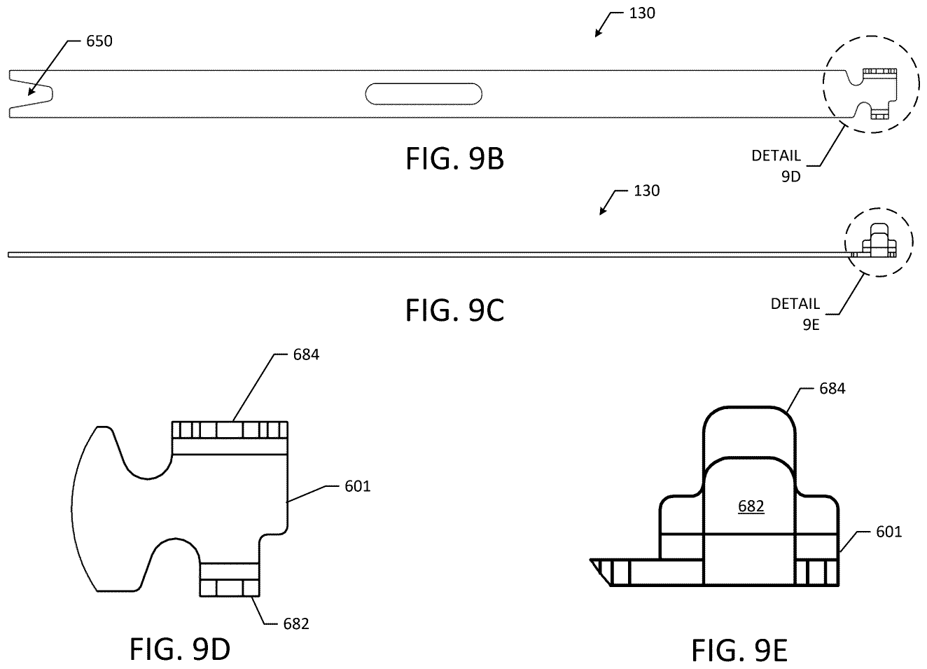

FIGS. 9A-9E illustrate an example jaw closer 130. The features and components of jaw closer 130 may have the same or similar construction and features as the jaw closer examples described in U.S. patent application Ser. No. 17/901,463 (see FIGS. 6A-6E and corresponding description of U.S. patent application Ser. No. 17/901,463). The jaw closer 130 may include an elongate base 610 with two drive flanges 682 and 684 that are configured to couple to respective ends of levers 184b and 184a (see FIG. 1B of U.S. patent application Ser. No. 17/901,463). The jaw closer 130 has a proximal end 601 and a distal end 603. Additionally, the jaw closer 130 may include a slot 640 that accommodates jaw anchor 190. The slot 640 is an oval slot that is adapted to limit the range of motion of the jaw closer 130 by providing a front-stop 642 and a backstop 644 for the jaw anchor 190. Specifically, the jaw closer 130 may move forward, towards the distal end 103 of the clip applier 100, until the jaw anchor 190 abuts the backstop 644. Similarly, the jaw closer 130 may move backward, toward the proximal end 101 of the clip applier 100, until the jaw anchor 190 abuts the front-stop 642 of the slot 640.

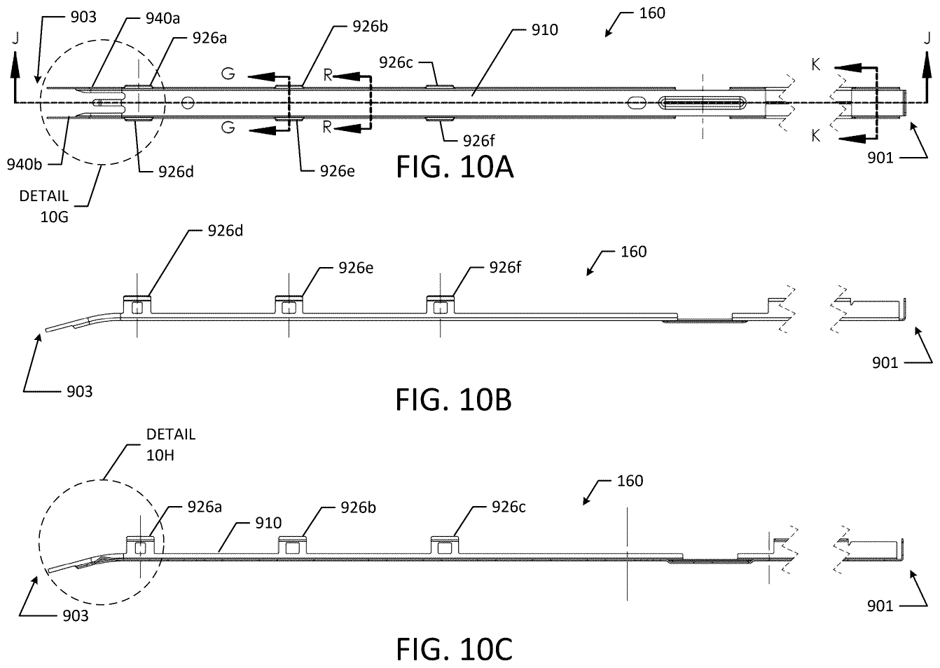

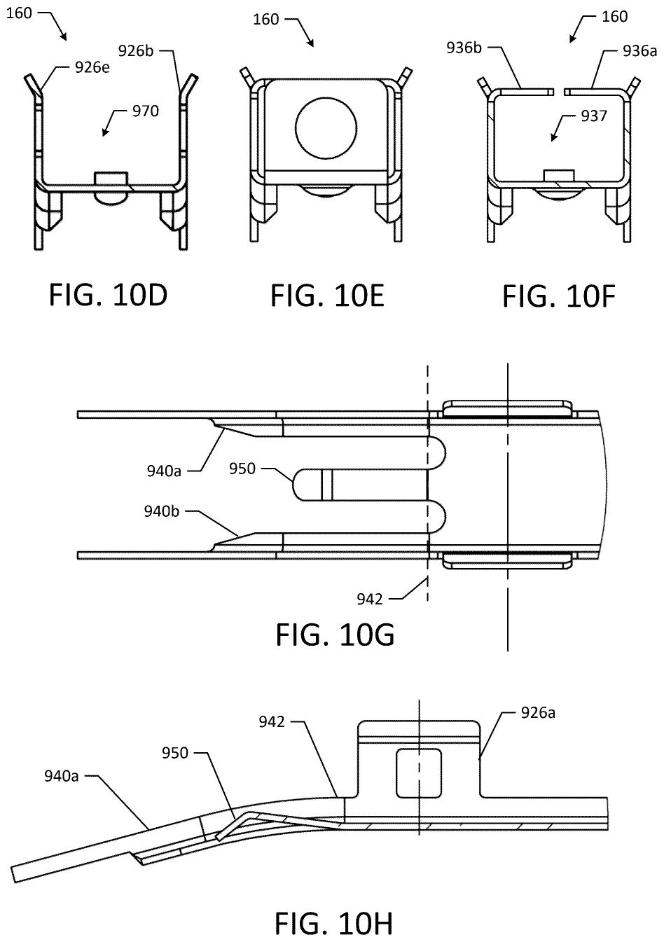

FIGS. 10A-10H illustrate an example dispenser 160. The features and components of dispenser 160 may have the same or similar construction and features as the dispenser examples described in U.S. patent application Ser. No. 17/901,463 (see FIGS. 9A-9N and corresponding description of U.S. patent application Ser. No. 17/901,463). The dispenser 160 may include an elongate base 910 with flanges 920a, 920b, 920c and 920d, which may serve as a securing flange or snap. For example, the flanges 920a-d may form a press-fit or snap-fit with corresponding structures on bottom housing 104. The dispenser 160 has a proximal end 901 and a distal end 903. At the distal end 903, the dispenser includes forks 940a and 940b that curve downward from the elongate base 910 at shoulder 942. Additionally, the dispenser 160 includes a retention prong 950 that curves upward from the elongate base 910 at the shoulder 942 (see FIGS. 9C and 9D). The forks 940a and 940b, hereinafter referred to generally as forks 940, create both a support surface and a track for the legs of a surgical clip 50.

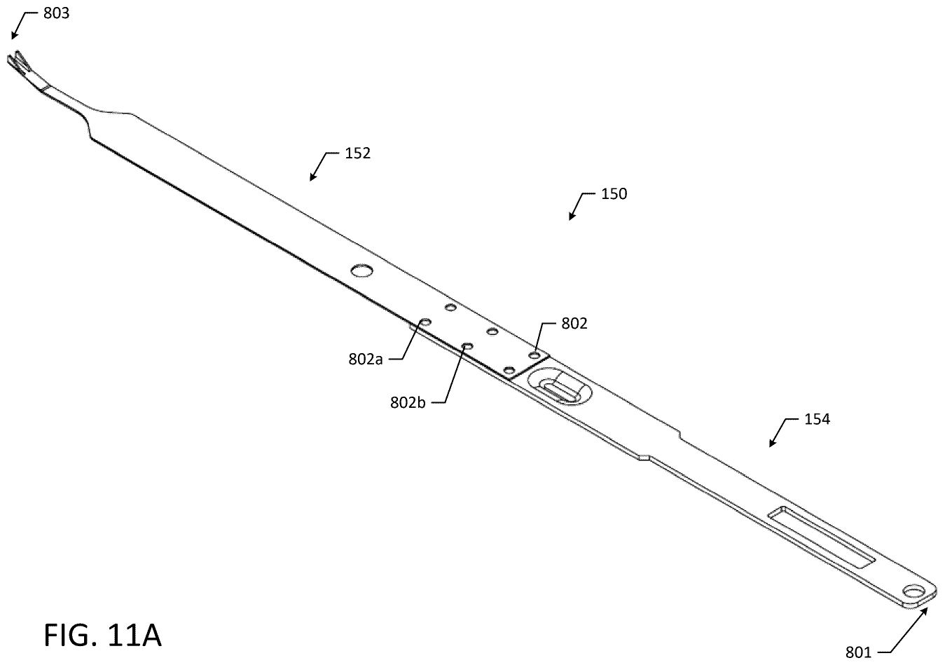

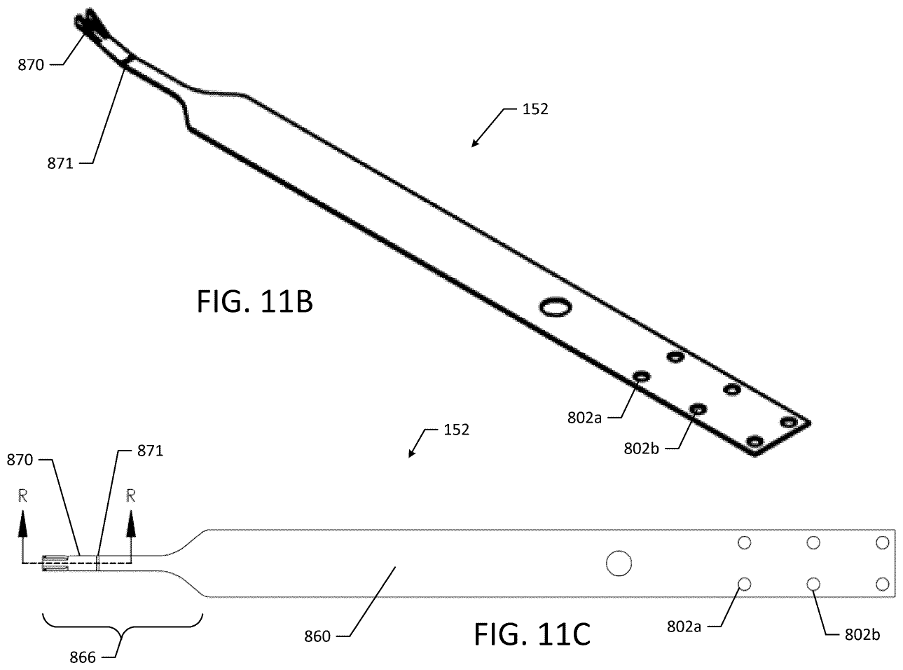

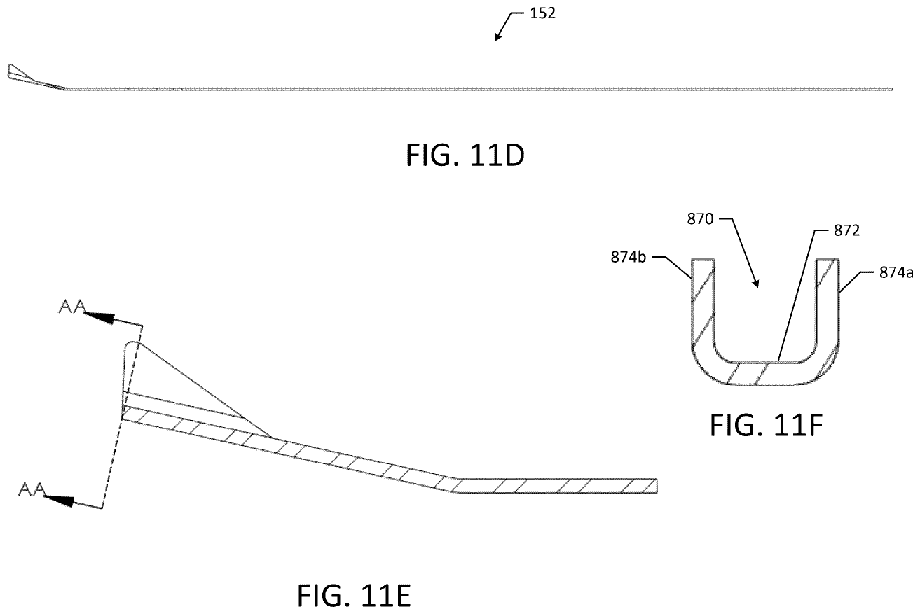

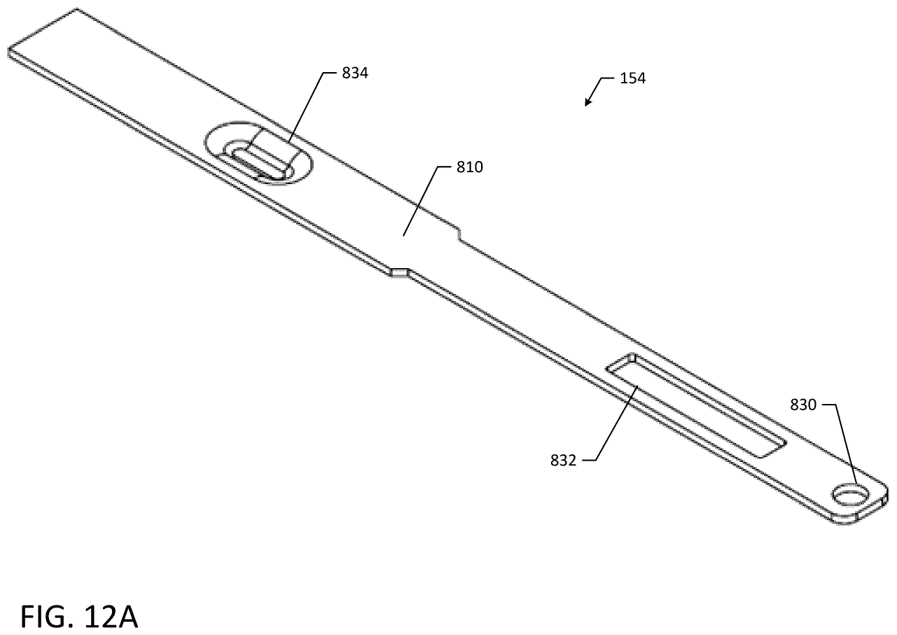

FIG. 11A illustrates an example clip loader assembly 150. The features and components of clip loader assembly 150 may have the same or similar construction and features as the dispenser examples described in U.S. patent application Ser. No. 17/901,463 (see FIGS. 8A, 8K, 8L, 8M and corresponding description of U.S. patent application Ser. No. 17/901,463). The clip loader assembly 150 may include a distal clip loader 152 and a proximal clip loader 154. The proximal clip loader 154 may be spot welded to the distal clip loader 152 at various locations or points (e.g., weld points 802a and 802b) within a weld region.

FIGS. 11B-11F illustrates an example distal clip loader 152. The features and components of distal clip loader 152 may have the same or similar construction and features as the distal clip loader examples described in U.S. patent application Ser. No. 17/901,463 (see FIGS. 8F-8H and corresponding description of U.S. patent application Ser. No. 17/901,463). The distal clip loader 152 may include an elongate base 860 and the end of the distal clip loader (e.g., the end corresponding to the distal end 803 of the clip loader assembly 150), includes a clip loading tip 870. Prior to reaching the clip loading tip 870, the elongate base 860 narrows at a clip loading portion 866 of the elongate base 860. Additionally, the clip loading portion 866 may curve upward at shoulder 871 until terminating at the clip loading tip 870. The clip loading portion 866 narrows to reduce the width of the elongate base 860, thereby allowing the distal clip loader 152, and more specifically the clip loading tip 870 to engage with individual surgical clips of a clip magazine. During application, the distal clip loader 152 pushes a leading surgical clip from the clip magazine over a retention prong 950 of the dispenser 160 (see FIGS. 9C and 9D and corresponding description of U.S. patent application Ser. No. 17/901,463).

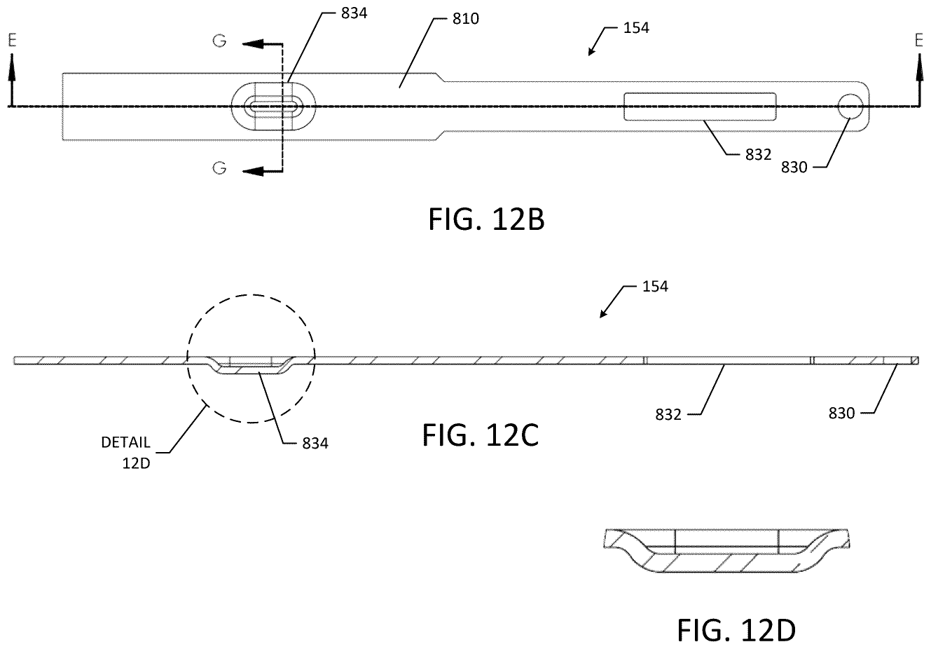

FIGS. 12A-12D illustrates an example proximal clip loader 154. The features and components of proximal clip loader 154 may have the same or similar construction and features as the proximal clip loader examples described in U.S. patent application Ser. No. 17/901,463 (see FIGS. 8B-8D, FIGS. 8N-8Q and corresponding description of U.S. patent application Ser. No. 17/901,463). The proximal clip loader 154 may include an elongate base 810 and drive aperture 830 that is configured to accommodate a drive flange (e.g., drive flange 1510, see FIG. 15A of U.S. patent application Ser. No. 17/901,463) of the proximal lever 182a of the right lever assembly 180a. For example, proximal clip loader 154 is positioned over the proximal lever 182a, such that the drive flange 1510 (see FIG. 15A of U.S. patent application Ser. No. 17/901,463) of the proximal lever 182a extends upwards through the drive aperture 830. Once the drive flange of the proximal lever 182a is positioned through the drive aperture 830, a corresponding thru-hole (e.g., thru-hole 1404, see FIG. 14A of U.S. patent application Ser. No. 17/901,463) of proximal lever 182b is positioned over the remaining portion of the drive flange 1510 that extends above the proximal clip loader 154. For example, the drive aperture 830 is aligned with the corresponding thru-hole 1404 of the proximal lever 182b. The proximal clip loader 154 also includes an alignment slot 832 as well as a vertical spacing and alignment protrusion 834.

It should be appreciated that some of the features and components of the surgical clip application device 100 are not described here in detail and instead may have the same construction and/or features as those described in U.S. patent application Ser. No. 17/901,463. Additionally, it should be appreciated that the examples described herein may include fewer (or in other examples more) components than the embodiments described in U.S. patent application Ser. No. 17/901,463. For instance, some examples of the clip application device 100 may be implemented without ratchet pawl 192 (e.g., the main rail 120 may be adapted for use without a ratchet pawl 192 and the jaw closer 130 may be without a pawl track 652).

Instead of the ratchet pawl 192, pawl pin 1102, etc. as illustrated and described in FIGS. 11A-11E and corresponding description of U.S. patent application Ser. No. 17/901,463, the clip applier 100 may operate without ratcheting or in a non-ratcheting configuration. By eliminating the unidirectional ratcheting (e.g., engaging handles to apply a surgical clip), the handles may be partially opened for repositioning and/or reorienting during a procedure, which may advantageously reduce injury, clip waste, and unintended errors during operations. For example, without being able to back-off or reduce pressure at the end of the clop application device, a clinician may inadvertently over tighten the clip prior to having the clip and tissue in the proper orientation.

It should be appreciated that for each component with multiple or alternative embodiments, each or any of the embodiments may include the same or similar features as a previously described or a later described embodiment. Specifically, any of the components or features illustrated in FIG. 1B, which are illustrated or described in more detail with relation to other figures, may be attributed to any of the components or features illustrated in FIG. 1C and vice versa.

Additionally, it should be appreciated that some example embodiments herein may include fewer or more components than other example embodiments. For instance, some example embodiments of the clip application device 100 herein can be implemented without one or more other component (e.g., pin, screw, etc.) used in other example embodiments of the clip application device 100.

Aspects of the subject matter described herein may be useful alone or in combination with one or more other aspects described herein. In an example, a surgical clip applying device includes an enclosure formed from a top housing, a bottom housing and a window cover. The clip applying device also includes a pair of handles including a left handle and a right handle, a main rail supporting a jaw closer, and a pair of clip applying jaws fixedly attached to the bottom housing and retained within the main rail. The pair of clip applying jaws has arms terminating with a pair of jaw heads. Additionally, the clip applying device includes a clip loader assembly with a clip loading tip at the clip loader assembly's distal end and a dispenser positioned above the clip loader assembly. The dispenser includes a pair of clip forks and a retention prong positioned between the pair of clip forks at the dispenser's distal end. The clip loading tip is configured to push a surgical clip supported by the pair of clip forks over the retention prong. The clip applying device also includes a clip pusher assembly configured to advance a plurality of clips towards the pair of jaw heads and a drive mechanism including a plurality of levers and at least one spring. The drive mechanism is configured to translate an actuation of the pair of handles to linear motion of the jaw closer and the clip loader assembly.

In another aspect of the present disclosure, the jaw closer includes an indent with cam members at its distal end. The cam members are configured to engage and compress corresponding ramps on the arms of the clip applying jaws thereby forcing the arms and the jaw heads together as the jaw closer is advanced towards the jaw heads.

To the extent that any of these aspects are mutually exclusive, it should be understood that such mutual exclusivity shall not limit in any way the combination of such aspects with any other aspect whether or not such aspect is explicitly recited. Any of these aspects may be claimed, without limitation, as a system, method, apparatus, device, medium, etc.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

The invention is claimed as follows:

1: A surgical clip applying device comprising:
an enclosure formed from a top housing, a bottom housing and a window cover;
a pair of handles including a left handle and a right handle each mounted for pivotal movement in the bottom housing;
a main rail fixedly mounted to the bottom housing within the enclosure;
a pair of clip applying jaws fixedly attached to the bottom housing and mounted through the main rail;
a jaw closer comprising a proximal end, a distal end, and an elongate base; and
a drive mechanism including a plurality of levers and at least one spring.

2: The surgical clip applying device of claim 1, wherein the enclosure includes an elongate base and upstanding sidewalls defining a U-shaped channel.

3: The surgical clip applying device of claim 1, wherein the pair of clip applying jaws include at least one arm, wherein the at least one arm terminates at a pair of spaced opposing jaw heads.

4: The surgical clip applying device of claim 1, wherein the pair of clip applying jaws include a pair of angled ramp surfaces disposed in said arms.

5: The surgical clip applying device of claim 4, wherein the arms are spaced inwardly from said jaw heads.

6: The surgical clip applying device of claim 1, wherein the distal end of the jaw closer includes a V-shaped indent.

7: The surgical clip applying device of claim 1, wherein the proximal end of the jaw closer includes a pair of upstanding drive flanges.

8: The surgical clip applying device of claim 1, wherein the device includes an elongate slot at a distance from the distal end of the jaw closer and at a distance from the proximal end of the jaw closer.

9: The surgical clip applying device of claim 1, wherein the jaw closer is disposed at a distance between the clip applying jaws and at a distance between the base of the rail.

10: The surgical clip applying device of claim 9, wherein the jaw closer is mounted for slideable movement in the channel toward and away from the jaw heads.

11: The surgical clip applying device of claim 1, wherein the device includes cam members configured to engage and compress a first ramp ramp surface and a second ramp surface, wherein the first ramp surface and the second ramp surface are disposed on the arms of the clip applying jaws.

12: The surgical clip applying device of claim 11, wherein the cam members are configured to force the arms and the jaw heads together as the jaw closer is advanced towards the jaw heads.

13: The surgical clip applying device of claim 1, wherein connecting the handles are engaged with the upstanding drive flanges of the jaw closer.

14: The surgical clip applying device of claim 1, wherein the drive mechanism is configured to translate an actuation of the pair of handles to a non-ratcheting linear motion of the jaw closer.

15: A clip applier surgery system comprising a clip applier, wherein the clip applier comprises:
an enclosure formed from a top housing, a bottom housing and a window cover;
a pair of handles including a left handle and a right handle each mounted for pivotal movement in the bottom housing;
a main rail fixedly mounted to the bottom housing within the enclosure;
a pair of clip applying jaws fixedly attached to the bottom housing and mounted through the main rail;
a jaw closer comprising a proximal end, a distal end, and an elongate base; and
a drive mechanism including a plurality of levers and at least one spring.

16: A method of ligating vessels during surgery using a surgical clip applying device, wherein the surgical applying device comprises:
an enclosure formed from a top housing, a bottom housing and a window cover;
a pair of handles including a left handle and a right handle each mounted for pivotal movement in the bottom housing;
a main rail fixedly mounted to the bottom housing within the enclosure;
a pair of clip applying jaws fixedly attached to the bottom housing and mounted through the main rail;
a jaw closer comprising a proximal end, a distal end, and an elongate base; and
a drive mechanism including a plurality of levers and at least one spring.

17: The method of claim 16, wherein the enclosure includes an elongate base and upstanding sidewalls defining a U-shaped channel.

18: The method of claim 16, wherein the pair of clip applying jaws include at least one arm, wherein the at least one arm terminates at a pair of spaced opposing jaw heads.

19: The method of claim 16, wherein the pair of clip applying jaws include a pair of angled ramp surfaces disposed in said arms.

20: The method of claim 16, wherein the arms are spaced inwardly from said jaw heads.
